(19) United States
(12) Patent Application Publication
(10) Pub. No.: US 2021/0239702 A1
(43) Pub. Date: Aug. 5, 2021

(54) METHODS FOR PREDICTING RESPONSIVENESS OF LUNG CANCER PATIENTS TO HER2-TARGETING THERAPIES

(71) Applicant: MEMORIAL SLOAN KETTERING CANCER CENTER, New York, NY (US)

(72) Inventors: Maurizio SCALTRITI, New York, NY (US); Bob T. LI, New York, NY (US)

(21) Appl. No.: 17/251,425

(22) PCT Filed: Jun. 13, 2019
(86) PCT No.: PCT/US2019/037112

§ 371 Date: Dec. 11, 2020

Related U.S. Application Data
(60) Provisional application No. 62/685,057, filed on Jun. 14, 2018.

(51) Int. Cl. G01N 33/574; G01N 33/50

(52) U.S. Cl. CPC G01N 33/57423; G01N 2333/71; G01N 33/5011

(57) ABSTRACT

The present disclosure provides methods for determining whether a patient diagnosed with lung cancer will benefit from or is predicted to be responsive to treatment with a therapeutic agent that targets HER2. These methods are based on detecting elevated levels of HER2 dimerization in a biological sample obtained from a lung cancer patient. Kits for use in practicing the methods are also provided.

### CROSS-REFERENCE TO RELATED PATENT APPLICATIONS

This application is a U.S. National Stage application under 35 U.S.C. § 371 of International Patent Application No. PCT/US2019/037112, filed Jun. 13, 2019, which claims the benefit of and priority to U.S. Provisional Appl. No. 62/685,057, filed Jun. 14, 2018, the disclosure of each of which is incorporated by reference herein in its entirety.

### TECHNICAL FIELD

The present disclosure relates to methods for determining whether a patient diagnosed with lung cancer will benefit from or is predicted to be responsive to treatment with a therapeutic agent that targets HER2. These methods are based on detecting elevated levels of HER2 dimerization in a biological sample obtained from a lung cancer patient. Kits for use in practicing the methods are also provided.

### BACKGROUND

Human epidermal growth factor receptor 2 (HER2, ERBB2) activating mutations occur in 2% of lung cancers. These mutations are transforming in lung cancer models and result in kinase activation, conferring some in vitro sensitivity to trastuzumab. An earlier generation of studies of trastuzumab in lung cancers selected patients based on HER2 protein expression by immunohistochemistry (IHC). Results were disappointing (Gatzemeier U, et al., Ann Oncol 15:19-27, 2004; Lara P N, Jr. et al., Clin Lung Cancer 5:231-6, 2004; Krug L M et al., Cancer 104:2149-55, 2005; Zinner R G et al., Lung Cancer 44:99-110, 2004; Langer C J et al., J Clin Oncol 22:1180-7, 2004; Clamon G et al., Cancer 103:1670-5, 2005). HER2 tyrosine kinase inhibitors dacomitinib, afatinib and neratinib have produced some responses in patients with HER2 mutant lung cancers, but the low response rates of 0-19% stalled further development (Li B T et al., Lung Cancer 90:617-9, 2015; Besse B et al., Ann Oncol 25, 2014; Gandhi L et al., Journal of Thoracic Oncology 12:S358-S359, 2016). Despite the plethora of agents targeting HER2 in patients with breast cancers, there is no approved targeted therapy for patients with HER2 mutant lung cancers.

### SUMMARY OF THE PRESENT TECHNOLOGY

In one aspect, the present disclosure provides a method for selecting lung cancer patients for treatment with a HER2-targeted therapeutic agent comprising: (a) detecting levels of HER2 dimerization in biological samples obtained from lung cancer patients; (b) identifying lung cancer patients that exhibit HER2 dimerization levels that are elevated compared to that observed in a healthy control subject or a predetermined threshold; and (c) administering a HER2-targeted therapeutic agent to the lung cancer patients of step (b). The lung cancer may be lung adenocarcinoma, squamous cell lung cancer, large cell lung cancer, or small cell lung cancer (SCLC). In some embodiments, the lung cancer patients harbor a HER2 mutation selected from the group consisting of exon 20 insYVMA, exon 20 insGSP, exon 20 insTGT, exon 20 insCPG, exon 20 G778_P780dup, exon 20 G776_V777>VCV, exon 20 G776delinsVC, L755A, L755S, L755P, V659E, S310F, and V777L. In certain embodiments, the lung cancer patients are human. Additionally or alternatively, in some embodiments of the methods disclosed herein, the biological samples are fresh tissue samples, frozen tissue samples, or fixed-formalin paraffin-embedded tissue samples.

Additionally or alternatively, in some embodiments of the methods disclosed herein, HER2 dimerization levels are detected via fluorescence resonance energy transfer (FRET), fluorescence lifetime imaging microscopy-fluorescence resonance energy transfer (FLIM-FRET), Western blotting, size exclusion chromatography, analytical ultracentrifugation, scattering techniques, NMR spectroscopy, isothermal titration calorimetry, fluorescence anisotropy, mass spectrometry, fluorescence correlation spectroscopy (FCS), fluorescence recovery after photobleaching, (FRAP), or proximity imaging (PRIM). HER2 dimerization may include HER-HER2 homodimerization and/or HER2-HER3 heterodimerization.

Additionally or alternatively, in some embodiments of the methods disclosed herein, the HER2-targeted therapeutic agent comprises a HER2 antibody-drug conjugate. In certain embodiments, the HER2 antibody-drug conjugate comprises trastuzumab, pertuzumab, margetuximab, or ertumaxomab. Additionally or alternatively, in some embodiments, the HER2 antibody-drug conjugate comprises an anthracycline, a microtubule inhibitor, a mitosis inhibitor, a topoisomerase inhibitor, a DNA damaging agent, a histone deacetylase inhibitor, a kinase inhibitor, a nucleotide analog, an amino acid analog, a vitamin analog, or an anti-metabolite. For example, the HER2 antibody-drug conjugate may include emtansine, deruxtecan, lapatinib, poziotinib, neratinib, and/or afatinib. Examples of HER2 antibody-drug conjugates include, but are not limited to, ado-trastuzumab emtansine (T-DM1), A166, ALT-P7, ARX788, DHES0815A, trastuzumab deruxtecan (DS-8201), DS-8201a, RC48, SYD985, MEDI4276 and XMT-1522.

Additionally or alternatively, in some embodiments of the methods disclosed herein, the lung cancer patients exhibit HER2 and/or HER3 expression levels that are elevated relative to that observed in a healthy control subject or a predetermined threshold. In other embodiments, the lung cancer patients exhibit HER2 and/or HER3 expression levels that are comparable to that observed in a healthy control subject or a predetermined threshold. In certain embodiments, HER2 and/or HER3 expression levels are measured using one or more of mass spectrometry, immunohistochemistry (IHC) or fluorescence in situ hybridization (FISH).

In another aspect, the present disclosure provides a method for treating lung cancer in a patient in need thereof comprising administering to the patient an effective amount of a HER2-targeted therapeutic agent, wherein the patient exhibits HER2 dimerization levels that are elevated compared to that observed in a healthy control subject or a predetermined threshold. The lung cancer may be lung adenocarcinoma, squamous cell lung cancer, large cell lung cancer, or small cell lung cancer (SCLC). In some embodiments, the patient harbors a HER2 mutation selected from the group consisting of exon 20 insYVMA, exon 20 insGSP, exon 20 insTGT, exon 20 insCPG, exon 20 G778_P780dup, exon 20 G776_V777>VCV, exon 20 G776delinsVC, L755A, L755S, L755P, V659E, S310F, and V777L. HER2 dimerization may include HER-HER2 homodimerization and/or HER2-HER3 heterodimerization.

Additionally or alternatively, in some embodiments of the methods disclosed herein, the HER2-targeted therapeutic agent comprises a HER2 antibody-drug conjugate. In certain embodiments, the HER2 antibody-drug conjugate comprises trastuzumab, pertuzumab, margetuximab, or ertumaxomab. Additionally or alternatively, in some embodiments, the HER2 antibody-drug conjugate comprises an anthracycline, a microtubule inhibitor, a mitosis inhibitor, a topoisomerase inhibitor, a DNA damaging agent, a histone deacetylase inhibitor, a kinase inhibitor, a nucleotide analog, an amino acid analog, a vitamin analog, or an anti-metabolite. For example, the HER2 antibody-drug conjugate may include emtansine, deruxtecan, lapatinib, poziotinib, neratinib, and/or afatinib. Examples of HER2 antibody-drug conjugates include, but are not limited to, ado-trastuzumab emtansine (T-DM1), A166, ALT-P7, ARX788, DHES0815A, trastuzumab deruxtecan (DS-8201), DS-8201a, RC48, SYD985, MEDI4276 and XMT-1522.

Additionally or alternatively, in some embodiments of the methods disclosed herein, the patient exhibits HER2 and/or HER3 expression levels that are elevated relative to that observed in a healthy control subject or a predetermined threshold. In other embodiments, the patient exhibits HER2 and/or HER3 expression levels that are comparable to that observed in a healthy control subject or a predetermined threshold. In certain embodiments, HER2 and/or HER3 expression levels are measured using one or more of mass spectrometry, immunohistochemistry (IHC) or fluorescence in situ hybridization (FISH).

Additionally or alternatively, in some embodiments, the methods of the present technology further comprise separately, sequentially or simultaneously administering to the patient at least one additional therapeutic agent. In certain embodiments, the at least one additional therapeutic agent is selected from the group consisting of immunotherapeutic agents, alkylating agents, topoisomerase inhibitors, endoplasmic reticulum stress inducing agents, antimetabolites, mitotic inhibitors, nitrogen mustards, nitrosoureas, alkylsulfonates, platinum agents, taxanes, vinca agents, anti-estrogen drugs, aromatase inhibitors, ovarian suppression agents, VEGF/VEGFR inhibitors, EGF/EGFR inhibitors, PARP inhibitors, cytostatic alkaloids, cytotoxic antibiotics, antimetabolites, endocrine/hormonal agents, bisphosphonate therapy agents, and phenphormin.

### BRIEF DESCRIPTION OF THE DRAWINGS

FIG. 1 shows the scheme used for the basket trial. CLIA, Clinical Laboratory Improvement Amendments; FISH, fluorescent in situ hybridization; IV, intravenous; NGS, next-generation sequencing; RECIST, Response Evaluation Criteria in Solid Tumors.

FIG. 2 shows characteristics of the patients enrolled in the basket trial.

FIG. 3 shows a waterfall plot of best response. RECIST, Response Evaluation Criteria in Solid Tumors.

FIG. 4 shows progression-free survival (PFS) of patients with HER2-mutant lung cancers (N=18). The median PFS for all patients was 5 months (95% CI, 3 to 9 months), and median PFS for the responders was 6 months (95% CI, 4 months to not reached).

FIG. 5 shows a swimmers plot of progression-free survival.

FIG. 6 shows treatment-related adverse events with total frequencies of >10%, according to common terminology criteria for adverse events version 4.1

FIG. 7 shows HER2 biomarker analysis.

FIG. 8 shows HER2 biomarker analysis of responders.

FIG. 9A shows a box plot illustrating the fluorescence resonance energy transfer (FRET) analysis showing HER2-HER3 heterodimerization in tissues from lung cancer patients. Distribution of FRET efficiency in each patient-derived tissue sample is plotted.

FIG. 9B shows the representative FRET images of a tissue sample from patient ID number 70 stained with anti-HER3 Alexa546 (donor) and with the combination of anti-HER3 Alexa546 with anti-HER2 Cy5 antibodies (donor⁺ acceptor). The FRET efficiency map shows yellow-red color in donor⁺ acceptor images and blue-green color in donor only images, indicating an interaction between HER2 and HER3 proteins. Scale bar, 50 μm.

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, microbiology and recombinant DNA are used. See, e.g., Sambrook and Russell eds. (2001) Molecular Cloning: A Laboratory Manual, 3rd edition; the series Ausubel et al., eds. (2007) Current Protocols in Molecular Biology; the series Methods in Enzymology (Academic Press, Inc., N.Y.); MacPherson et al., (1991) PCR 1: A Practical Approach (IRL Press at Oxford University Press); MacPherson et al., (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al., eds (1996) Weir's Handbook of Experimental Immunology.

HER2-targeted therapy in lung cancers has traditionally focused on HER2 protein expression, which was driven in part by observations in breast cancers that trastuzumab binding requires HER2 protein overexpression to elicit antitumor activity through inhibition of ligand independent HER2 signaling, receptor internalization, and antibody dependent cell mediated cytotoxicity (Slamon D J et al., N Engl J Med 344:783-92, 2001; Hudis C A, N Engl J Med 357:39-51, 2007). However, HER2 IHC 3+ or HER2 amplification are much rarer in lung tumors than in breast cancers (2% vs 20%). Indeed, clinical trials testing the activity of trastuzumab in lung cancers were conducted in tumors with lower levels of HER2 IHC positivity and/or were not driven by HER2 signaling. The results of six phase 2 trials of trastuzumab in HER2 IHC positive lung cancers were uniformly negative, and more recent studies of ado-trastuzumab emtansine have again confirmed that HER2 IHC is not the ideal biomarker in lung cancers.

The present disclosure demonstrates that lung cancer patients exhibiting elevated HER2 dimerization levels show increased responsiveness to HER2 antibody-drug conjugates. Out of 18 patients with ERBB2-mutant lung cancers, 8 experienced confirmed radiologic partial response to ado-trastuzumab emtansine therapy and, in several additional patients, the disease was controlled over time. Moreover, of the responding patients, only one had a tumor with concomitant ERBB2 amplification/HER2 overexpression. Such a small overlap demonstrates that HER2 mutation and amplification are largely separate therapeutic targets. As demonstrated herein, the low or undetectable levels of HER2 protein expression (confirmed by mass spectrometry) among HER2 mutants and the observed responsiveness to ado-trastuzumab emtansine support an alternative mechanism for binding and internalization of the antibody drug conjugate that is independent of HER2 protein overexpression. Without wishing to be bound by theory, it is believed that HER2 activating mutations in lung cancers exhibit increased dimer formation (e.g., HER2-HER2 homodimers or HER2 heterodimers), which may consequently increase preferential binding and internalization of HER2 antibody-drug conjugates.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function.

Administration can be carried out by any suitable route, including orally, intranasally, parenterally (intravenously, intramuscularly, intraperitoneally, or subcutaneously), or topically. Administration includes self-administration and the administration by another.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. As used herein, “antibodies” include intact immunoglobulins and antigen binding fragments thereof, which specifically bind to a molecule of interest (or a group of highly similar molecules of interest) to the substantial exclusion of binding to other molecules (for example, antibodies and antibody fragments that have a binding constant for the molecule of interest that is at least 10³ M-1 greater, at least 10⁴ M-1 greater or at least 10⁵ M-1 greater than a binding constant for other molecules in a biological sample). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3rd Ed., W.H. Freeman & Co., New York, 1997.

As used herein, the term “conjugated” refers to the association of two molecules by any method known to those in the art. Suitable types of associations include chemical bonds and physical bonds. Chemical bonds include, for example, covalent bonds and coordinate bonds. Physical bonds include, for instance, hydrogen bonds, dipolar interactions, van der Waal forces, electrostatic interactions, hydrophobic interactions and aromatic stacking.

The terms “complementary” or “complementarity” as used herein with reference to polynucleotides (i.e., a sequence of nucleotides such as an oligonucleotide or a target nucleic acid) refer to the base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5′-A-G-T-3′” is complementary to the sequence “3′-T-C-A-S.” Certain bases not commonly found in naturally-occurring nucleic acids may be included in the nucleic acids described herein. These include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complementary sequence can also be an RNA sequence complementary to the DNA sequence or its complementary sequence, and can also be a cDNA.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease or condition, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of lung cancer (e.g. HER2 mutant lung cancer). As used herein, a “therapeutically effective amount” of a composition refers to composition levels in which the physiological effects of a disease or condition are ameliorated or eliminated. A therapeutically effective amount can be given in one or more administrations.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the term “gene” means a segment of DNA that contains all the information for the regulated biosynthesis of an RNA product, including promoters, exons, introns, and other untranslated regions that control expression.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same nucleobase or amino acid, then the molecules are homologous at that position. A degree of homology between sequences is a function of the number of matching or homologous positions shared by the sequences. A polynucleotide or polynucleotide region (or a polypeptide or polypeptide region) has a certain percentage (for example, at least 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art. In some embodiments, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the National Center for Biotechnology Information. Biologically equivalent polynucleotides are those having the specified percent homology and encoding a polypeptide having the same or similar biological activity. Two sequences are deemed “unrelated” or “non-homologous” if they share less than 40% identity, or less than 25% identity, with each other.

The term “hybridize” as used herein refers to a process where two substantially complementary nucleic acid strands (at least about 65% complementary over a stretch of at least 14 to 25 nucleotides, at least about 75%, or at least about 90% complementary) anneal to each other under appropriately stringent conditions to form a duplex or heteroduplex through formation of hydrogen bonds between complementary base pairs. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Hybridization and the strength of hybridization (i.e., the strength of the association between the nucleic acids) is influenced by such factors as the degree of complementarity between the nucleic acids, stringency of the conditions involved, and the thermal melting point (Tm) of the formed hybrid. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al., 1994, Current Protocols in Molecular Biology, John Wiley & Sons, Secaucus, N.J. In some embodiments, specific hybridization occurs under stringent hybridization conditions. An oligonucleotide or polynucleotide (e.g., a probe or a primer) that is specific for a target nucleic acid will “hybridize” to the target nucleic acid under suitable conditions.

As used herein, “oligonucleotide” refers to a molecule that has a sequence of nucleic acid bases on a backbone comprised mainly of identical monomer units at defined intervals. The bases are arranged on the backbone in such a way that they can bind with a nucleic acid having a sequence of bases that are complementary to the bases of the oligonucleotide. The most common oligonucleotides have a backbone of sugar phosphate units. A distinction may be made between oligodeoxyribonucleotides that do not have a hydroxyl group at the 2′ position and oligoribonucleotides that have a hydroxyl group at the 2′ position.

Oligonucleotides may also include derivatives, in which the hydrogen of the hydroxyl group is replaced with organic groups, e.g., an allyl group. One or more bases of the oligonucleotide may also be modified to include a phosphorothioate bond (e.g., one of the two oxygen atoms in the phosphate backbone which is not involved in the internucleotide bridge, is replaced by a sulfur atom) to increase resistance to nuclease degradation. The exact size of the oligonucleotide will depend on many factors, which in turn depend on the ultimate function or use of the oligonucleotide. The oligonucleotide may be generated in any manner, including, for example, chemical synthesis, DNA replication, restriction endonuclease digestion of plasmids or phage DNA, reverse transcription, PCR, or a combination thereof. The oligonucleotide may be modified e.g., by addition of a methyl group, a biotin or digoxigenin moiety, a fluorescent tag or by using radioactive nucleotides.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration. Pharmaceutically-acceptable carriers and their formulations are known to one skilled in the art and are described, for example, in Remington's Pharmaceutical Sciences (20th edition, ed. A. Gennaro, 2000, Lippincott, Williams & Wilkins, Philadelphia, Pa.).

As used herein, the term “polynucleotide” or “nucleic acid” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein, “prevention,” “prevent,” or “preventing” of a disorder or condition refers to one or more compounds that, in a statistical sample, reduces the occurrence of the disorder or condition in the treated sample relative to an untreated control sample, or delays the onset of one or more symptoms of the disorder or condition relative to the untreated control sample. As used herein, preventing a HER2 mutant cancer (e.g. HER2 mutant lung cancer), includes preventing or delaying the initiation of symptoms of a HER2 mutant cancer (e.g. HER2 mutant lung cancer). As used herein, prevention of a HER2 mutant cancer (e.g. HER2 mutant lung cancer) also includes preventing a recurrence of one or more signs or symptoms of a HER2 mutant cancer (e.g. HER2 mutant lung cancer).

“RECIST” shall mean an acronym that stands for “Response Evaluation Criteria in Solid Tumors” and is a set of published rules that define when cancer patients improve (“respond”), stay the same (“stable”) or worsen (“progression”) during treatments. Response as defined by RECIST criteria have been published, for example, at Journal of the National Cancer Institute, Vol. 92, No. 3, Feb. 2, 2000 and RECIST criteria can include other similar published definitions and rule sets. One skilled in the art would understand definitions that go with RECIST criteria, as used herein, such as “Partial Response (PR),” “Complete Response (CR),” “Stable Disease (SD)” and “Progressive Disease (PD).”

The irRECIST overall tumor assessment is based on total measurable tumor burden (TMTB) of measured target and new lesions, non-target lesion assessment and new non-measurable lesions. At baseline, the sum of the longest diameters (SumD) of all target lesions (up to 2 lesions per organ, up to total 5 lesions) is measured. At each subsequent tumor assessment (TA), the SumD of the target lesions and of new, measurable lesions (up to 2 new lesions per organ, total 5 new lesions) are added together to provide the TMTB.

Overall Assessments by irRECIST

| | |
|---|---|
| Complete | Complete disappearance of all measurable and non- |
| Response | measurable lesions. Lymph nodes must decrease to <10 |
| (irCR) | mm in short axis |
| Partial | Decrease of ≥30% in TMTB relative to baseline, non-target |
| Response | lesions are irNN, and no unequivocal progression of new |
| (irPR) | nonmeasurable lesions |
| | If new measurable lesions appear in subjects with no |
| | target lesions at baseline, irPD will be assessed. |
| | That irPD time point will be considered a new |
| | baseline, and all subsequent time points will be |
| | compared to it for response assessment. irPR is |
| | possible if the TMTB of new measurable lesions |
| | decreases by ≥30% compared to the first irPD |
| | documentation |
| | irRECIST can be used in the adjuvant setting, in |
| | subjects with no visible disease on CT/MRI scans. |
| | The appearance of new measurable lesion(s) |
| | automatically leads to an increase in TMTB by 100% |
| | and leads to irPD. These subjects can achieve a |
| | response if the TMTB decreases at follow-up, as a |
| | sign of delayed response. |
| | Based on the above, sponsors may consider enrolling |
| | subjects with no measurable disease and/or no visible |
| | disease in studies with response related endpoints. |
| Stable | Failure to meet criteria for irCR or irPR in the absence of |
| Disease | irPD |
| (irSD) | |
| Progressive | Minimum 20% increase and minimum 5 mm absolute |
| Disease | increase in TMTB compared to nadir, or irPD for non-target |
| (irPD) | or new non-measurable lesions. Confirmation of progression |
| | is recommended minimum 4 weeks after the first irPD |
| | assessment. An irPD confirmation scan may be |
| | recommended for subjects with a minimal TMTB %- |
| | increase over 20% and especially during the flare time- |
| | window of the first 12 weeks of treatment, depending on the |
| | compound efficacy expectations, to account for expected |
| | delayed response. |
| | In irRECIST a substantial and unequivocal increase |
| | of non-target lesions is indicative of progression. |
| | IrPD may be assigned for a subject with multiple new |
| | non-measurable lesions if they are considered to be |
| | a sign of unequivocal massive worsening |
| Other | irNE: used in exceptional cases where insufficient data exist. |
| | irND: in adjuvant setting when no disease is detected |
| | irNN:, no target disease was identified at baseline, and at |
| | follow-up the subject fails to meet criteria for irCR or irPD |

As used herein, the term “sample” refers to clinical samples obtained from a subject. Biological samples may include tissues, cells, protein or membrane extracts of cells, mucus, sputum, bone marrow, bronchial alveolar lavage (BAL), bronchial wash (BW), and biological fluids (e.g., ascites fluid or cerebrospinal fluid (CSF)) isolated from a subject, as well as tissues, cells and fluids (blood, plasma, saliva, urine, serum etc.) present within a subject.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, the terms “subject,” “individual,” or “patient” are used interchangeably and refer to an individual organism, a vertebrate, a mammal, or a human. In certain embodiments, the individual, patient or subject is a human.

“Treating”, “treat”, or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment or prevention of medical diseases and conditions as described are intended to mean “substantial,” which includes total but also less than total treatment or prevention, and wherein some biologically or medically relevant result is achieved. The treatment may be a continuous prolonged treatment for a chronic disease or a single, or few time administrations for the treatment of an acute condition.

### HER2

HER2 (ErbB-2, c-erbB2 or Her2/neu) is a member of the human epidermal growth factor receptor family (which includes HER1 (Epidermal Growth Factor Receptor-EGFR, or ErbB1), HER3 (ErbB3) and HER4 (ErbB4)). HER2 is a proto-oncogene that encodes a 185-kDa plasma membrane-bound tyrosine kinase receptor, located on chromosome 17 at q21. Unlike HER1, HER3 and HER4, HER2 is classified as an orphan receptor, i.e., no direct ligand for HER2 has been discovered. HER2 stimulation by extracellular signals leads to the activation of downstream pathways such as mitogen-activated protein kinase (MAPK), phosphoinositide-3-kinase (PI3K), phospholipase C and protein kinase C, thereby inducing signal transduction and transcription. As such, HER2 gene amplification and protein overexpression are associated with the pathogenesis and progression of several human cancers, and are often viewed as indicators of poor prognosis. HER2/neu overexpression has been reported in many epithelial malignancies including lung, prostate, bladder, pancreatic cancer and osteosarcoma. Due to the revolutionary impact of anti-HER2 therapy in breast cancer patients, the role of HER2 has been evaluated in other tumor types. e.g., Tapia et al., Mod Pathol. 20(2):192-8 (2007). However, studies have shown that HER2 blockade in non-breast cancers was not as successful as the breast cancer clinical trials. Clamon et al., Cancer 103(8):1670-5 (2005); Harder et al., Br J Cancer 106(6):1033-8 (2012); Hecht et al., J Clin Oncol. 34(5):443-51 (2016); Michaelson et al., Int J Radiat Oncol Biol Phys. 97(5): 995-1001 (2017); Rubinson et al., Invest New Drugs 32(1): 113-122 (2014); and Satoh et al., J Clin Oncol. 32(19):2039-49 (2014).

### HER2-Targeted Therapeutic Agents and Methods of the Present Technology

As used herein, a “HER2 antibody-drug conjugate” refers to a HER2-targeting antibody that is conjugated to a drug that has a cytotoxic or cytostatic effect on cancer cells. In some embodiments, the drug is: (i) a chemotherapeutic agent, which may function as microtubule inhibitor, a mitosis inhibitor, a topoisomerase inhibitor, a DNA damaging agent, a histone deacetylase inhibitor, a kinase inhibitor, a nucleotide analog, an amino acid analog, a vitamin analog, an antimetabolite (e.g., folic acid analogs); (ii) a protein toxin, which may function enzymatically; and/or (iii) a radioisotope. Non-limiting examples of suitable chemotherapeutic agents include alkylating agents such a thiotepa and cyclosphosphamide; alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, triethylenethiophosphoramide, and trimethylolomelamine; acetogenins (especially bullatacin andbullatacinone); camptothecin (including the synthetic analoguetopotecan); bryostatin; callystatin; CC-1065 (including its adozelesin, carzelesin, and bizelesin synthetic analogues); cryptophycines (particularly cryptophycin 1 and cryptophycin 8); dolastatin, auristatins, (including analogues monomethyl-auristatin E andmonomethyl-auristatin F); duocarmycin (including the synthetic analogues, KW-2189 and CBI-TMI); eleutherobin; pancratistatin; sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine; trofosfamide, uracil mustard; nitrosureassuch as carmustine, chlorozotocin, fotemustine, lomustine, nimustine, ranimustine; antibiotics such as the enediyne antibiotics (e.g., calicheamicin such as calichemicin yl); dynemicin, including dynemicin A; bisphosphonates, such as clodronate; esperamicin; as well as neocarzinostatin chromophore and related chromoproteinenediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin, and deoxydoxorubicin), epirubucin, esorubicin, idarubicin, marcellomycin, mitomycins such as mitomycin C, mycophenolicacid, nogalamycin, olivomycins, peplomycin, potfiromycin, puromycine, quelamycin, rodorubicin, streptonigrin, streptozocin, tubercidin, ubenimex, zinostatin, zorubicin; anti-metabolites such a methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adranals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; democolcine; diaziquone; elfomithine; elliptinium acetate; an epothilone; etoglucid; gallium nitrate; hydroxyurea; lentinan; lonidamine; maytansinoids such asmaytansine and ansamitocins; mitoguazone, mitoxantrone; mopidamol; nitracrine; pentostatin; phenamet; pirarubicin; losoxantrone; podophyllinic acid; 2-ethylhydrazide; procarbazine; PSK®; razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitabronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids, or derivatives of any of the above. Also included in this definition are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene; aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, and goserelin; and pharmaceutically acceptable salts, acids, or derivatives of any of the above.

Examples of antimetabolites include 5-fluorouracil (5-FU), 6-mercaptopurine (6-MP), capecitabine, cytarabine, floxuridine, fludarabine, gemcitabine, hydroxyurea, methotrexate, pemetrexed, and mixtures thereof.

Examples of topoisomerase I inhibitors include SN-38, ARC, NPC, camptothecin, topotecan, 9-nitrocamptothecin, exatecan, lurtotecan, lamellarin D9-aminocamptothecin, rubifen, gimatecan, diflomotecan, BN80927, DX-8951f, MAG-CPT, and mixtures thereof. Examples of topoisomerase II inhibitors include amsacrine, etoposide, etoposide phosphate, teniposide, daunorubicin, mitoxantrone, amsacrine, ellipticines, aurintricarboxylic acid, doxorubicin, and HU-331 and combinations thereof.

Examples of microtubule inhibitors include taxanes, vinca alkaloids, emtansine, deruxtecan, colchicine, podophyllotoxin, estramustine, nocodazole, etc.

Examples of taxanes include paclitaxel, docetaxel, accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, and mixtures thereof.

Examples of DNA alkylating agents include cyclophosphamide, chlorambucil, melphalan, bendamustine, uramustine, estramustine, carmustine, lomustine, nimustine, ranimustine, streptozotocin; busulfan, mannosulfan, and mixtures thereof.

In one aspect, the present disclosure provides a method for selecting lung cancer patients for treatment with a HER2-targeted therapeutic agent comprising: (a) detecting levels of HER2 dimerization in biological samples obtained from lung cancer patients; (b) identifying lung cancer patients that exhibit HER2 dimerization levels that are elevated compared to that observed in a healthy control subject or a predetermined threshold; and (c) administering a HER2-targeted therapeutic agent to the lung cancer patients of step (b). The lung cancer may be lung adenocarcinoma, squamous cell lung cancer, large cell lung cancer, or small cell lung cancer (SCLC). In some embodiments, the lung cancer patients harbor a HER2 mutation selected from the group consisting of exon 20 insYVMA, exon 20 insGSP, exon 20 insTGT, exon 20 insCPG, exon 20 G778_P780dup, exon 20 G776_V777>VCV, exon 20 G776delinsVC, L755A, L755S, L755P, V659E, S310F, and V777L. In certain embodiments, the lung cancer patients are human. Additionally or alternatively, in some embodiments of the methods disclosed herein, the biological samples are fresh tissue samples, frozen tissue samples, or fixed-formalin paraffin-embedded tissue samples.

Additionally or alternatively, in some embodiments of the methods disclosed herein, HER2 dimerization levels are detected via fluorescence resonance energy transfer (FRET), fluorescence lifetime imaging microscopy-fluorescence resonance energy transfer (FLIM-FRET), Western blotting, size exclusion chromatography, analytical ultracentrifugation, scattering techniques, NMR spectroscopy, isothermal titration calorimetry, fluorescence anisotropy, mass spectrometry, fluorescence correlation spectroscopy (FCS), fluorescence recovery after photobleaching, (FRAP), or proximity imaging (PRIM). HER2 dimerization may include HER-HER2 homodimerization and/or heterodimerization with other members of the HER-family, such as HER3.

Additionally or alternatively, in some embodiments of the methods disclosed herein, the HER2-targeted therapeutic agent comprises a HER2 antibody-drug conjugate. In certain embodiments, the HER2 antibody-drug conjugate comprises trastuzumab, pertuzumab, margetuximab, or ertumaxomab. Additionally or alternatively, in some embodiments, the HER2 antibody-drug conjugate comprises an anthracycline, a microtubule inhibitor, a mitosis inhibitor, a topoisomerase inhibitor, a DNA damaging agent, a histone deacetylase inhibitor, a kinase inhibitor, a nucleotide analog, an amino acid analog, a vitamin analog, or an anti-metabolite.

For example, the HER2 antibody-drug conjugate may include emtansine, deruxtecan, lapatinib, poziotinib, neratinib, and/or afatinib. Examples of HER2 antibody-drug conjugates include, but are not limited to, ado-trastuzumab emtansine (T-DM1), A166, ALT-P7, ARX788, DHES0815A, trastuzumab deruxtecan (DS-8201), DS-8201a, RC48, SYD985, MEDI4276 and XMT-1522.

Additionally or alternatively, in some embodiments of the methods disclosed herein, the lung cancer patients exhibit HER2 and/or HER3 expression levels that are elevated relative to that observed in a healthy control subject or a predetermined threshold. In other embodiments, the lung cancer patients exhibit HER2 and/or HER3 expression levels that are comparable to that observed in a healthy control subject or a predetermined threshold. In certain embodiments, HER2 and/or HER3 expression levels are measured using one or more of mass spectrometry, immunohistochemistry (IHC) or fluorescence in situ hybridization (FISH).

In another aspect, the present disclosure provides a method for treating lung cancer in a patient in need thereof comprising administering to the patient an effective amount of a HER2-targeted therapeutic agent, wherein the patient exhibits HER2 dimerization levels that are elevated compared to that observed in a healthy control subject or a predetermined threshold. The lung cancer may be lung adenocarcinoma, squamous cell lung cancer, large cell lung cancer, or small cell lung cancer (SCLC). In some embodiments, the patient harbors a HER2 mutation selected from the group consisting of exon 20 insYVMA, exon 20 insGSP, exon 20 insTGT, exon 20 insCPG, exon 20 G778_P780dup, exon 20 G776_V777>VCV, exon 20 G776delinsVC, L755A, L755S, L755P, V659E, S310F, and V777L. HER2 dimerization may include HER-HER2 homodimerization and/or heterodimerization with other members of the HER-family, such as HER3.

Additionally or alternatively, in some embodiments of the methods disclosed herein, the HER2-targeted therapeutic agent comprises a HER2 antibody-drug conjugate. In certain embodiments, the HER2 antibody-drug conjugate comprises trastuzumab, pertuzumab, margetuximab, or ertumaxomab. Additionally or alternatively, in some embodiments, the HER2 antibody-drug conjugate comprises an anthracycline, a microtubule inhibitor, a mitosis inhibitor, a topoisomerase inhibitor, a DNA damaging agent, a histone deacetylase inhibitor, a kinase inhibitor, a nucleotide analog, an amino acid analog, a vitamin analog, or an anti-metabolite. For example, the HER2 antibody-drug conjugate may include emtansine, deruxtecan, lapatinib, poziotinib, neratinib, and/or afatinib. Examples of HER2 antibody-drug conjugates include, but are not limited to, ado-trastuzumab emtansine (T-DM1), A166, ALT-P7, ARX788, DHES0815A, trastuzumab deruxtecan (DS-8201), DS-8201a, RC48, SYD985, MEDI4276 and XMT-1522.

Additionally or alternatively, in some embodiments of the methods disclosed herein, the patient exhibits HER2 and/or HER3 expression levels that are elevated relative to that observed in a healthy control subject or a predetermined threshold. In other embodiments, the patient exhibits HER2 and/or HER3 expression levels that are comparable to that observed in a healthy control subject or a predetermined threshold. In certain embodiments, HER2 and/or HER3 expression levels are measured using one or more of mass spectrometry, immunohistochemistry (IHC) or fluorescence in situ hybridization (FISH).

### Modes of Administration and Effective Dosages

Any method known to those in the art for contacting a cell, organ or tissue with one or more HER2-targeted therapeutic agents disclosed herein may be employed. Suitable methods include in vitro, ex vivo, or in vivo methods. In vivo methods typically include the administration of one or more HER2-targeted therapeutic agents to a mammal, suitably a human. When used in vivo for therapy, the one or more HER2-targeted therapeutic agents described herein are administered to the subject in effective amounts (i.e., amounts that have desired therapeutic effect). The dose and dosage regimen will depend upon the degree of the disease state of the subject, the characteristics of the particular HER2-targeted therapeutic agent used, e.g., its therapeutic index, and the subject's history.

The effective amount may be determined during pre-clinical trials and clinical trials by methods familiar to physicians and clinicians. An effective amount of one or more HER2-targeted therapeutic agents useful in the methods may be administered to a mammal in need thereof by any of a number of well-known methods for administering pharmaceutical compounds. The HER2-targeted therapeutic agent may be administered systemically or locally.

The one or more HER2-targeted therapeutic agents described herein can be incorporated into pharmaceutical compositions for administration, singly or in combination, to a subject for the treatment or prevention of a HER2 mutant cancer (e.g. HER2 mutant lung cancer), and/or a subject for the treatment or prevention of HER2 mutant cancer (e.g. HER2 mutant lung cancer). Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided in a kit containing all necessary equipment (e.g., vials of drug, vials of diluent, syringes and needles) for a treatment course (e.g., 7 days of treatment).

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The pharmaceutical compositions having one or more HER2-targeted therapeutic agents disclosed herein can include a carrier, which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like.

Glutathione and other antioxidants can be included to prevent oxidation. In many cases, it will be advantageous to include isotonic agents, for example, sugars, polyalcohols such as mannitol, sorbitol, or sodium chloride in the composition. Prolonged absorption of the injectable compositions can be brought about by including in the composition an agent that delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressurized container or dispenser, which contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

A therapeutic agent can be formulated in a carrier system. The carrier can be a colloidal system. The colloidal system can be a liposome, a phospholipid bilayer vehicle, or a lipid nanoparticle. In one embodiment, the therapeutic agent is encapsulated in a liposome while maintaining the agent's structural integrity. One skilled in the art would appreciate that there are a variety of methods to prepare liposomes. (See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother., 34(7-8):915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic agent can be embedded in the polymer matrix, while maintaining the agent's structural integrity. The polymer may be natural, such as polypeptides, proteins or polysaccharides, or synthetic, such as poly α-hydroxy acids.

Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compounds can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4(3):201-9 (1994); and Gregoriadis, “Engineering Liposomes for Drug Delivery: Progress and Problems,” Trends Biotechnol., 13(12):527-37 (1995). Mizguchi, et al., Cancer Lett., 100:63-69 (1996), describes the use of fusogenic liposomes to deliver a protein to cells both in vivo and in vitro.

Dosage, toxicity and therapeutic efficacy of any therapeutic agent can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans accurately. Levels in plasma may be measured, for example, by high performance liquid chromatography.

Typically, an effective amount of the one or more HER2-targeted therapeutic agents disclosed herein sufficient for achieving a therapeutic or prophylactic effect, range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example, dosages can be 1 mg/kg body weight or 10 mg/kg body weight every day, every two days or every three days or within the range of 1-10 mg/kg every week, every two weeks or every three weeks. In one embodiment, a single dosage of the therapeutic compound ranges from 0.001-10,000 micrograms per kg body weight. In one embodiment, one or more HER2-targeted therapeutic agent concentrations in a carrier range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

In some embodiments, a therapeutically effective amount of one or more HER2-targeted therapeutic agents may be defined as a concentration of inhibitor at the target tissue of 10⁻³² to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue, such as by single daily or weekly administration, but also including continuous administration (e.g., parenteral infusion or transdermal application).

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance with the present methods can be any mammal, including, for example, farm animals, such as sheep, pigs, cows, and horses; pet animals, such as dogs and cats; laboratory animals, such as rats, mice and rabbits. In some embodiments, the mammal is a human.

### Combination Therapy

In some embodiments, one or more of the HER2-targeted therapeutic agents disclosed herein may be combined with one or more additional therapies for the prevention or treatment of lung cancer (e.g. HER2 mutant lung cancer). Additional therapeutic agents include, but are not limited to, ABRAXANE® (albumin-bound paclitaxel), GEMZAR® (gemcitabine), 5-FU (fluorouracil), ONIVYDE® (irinotecan liposome injection), surgery, radiation, or a combination thereof.

In some embodiments, the one or more HER2-targeted therapeutic agents disclosed herein may be separately, sequentially or simultaneously administered with at least one additional therapeutic agent. In certain embodiments, the at least one additional therapeutic agent is selected from the group consisting of immunotherapeutic agents, alkylating agents, topoisomerase inhibitors, endoplasmic reticulum stress inducing agents, antimetabolites, mitotic inhibitors, nitrogen mustards, nitrosoureas, alkylsulfonates, platinum agents, taxanes, vinca agents, anti-estrogen drugs, aromatase inhibitors, ovarian suppression agents, VEGF/VEGFR inhibitors, EGF/EGFR inhibitors, PARP inhibitors, cytostatic alkaloids, cytotoxic antibiotics, antimetabolites, endocrine/hormonal agents, bisphosphonate therapy agents, phenphormin and targeted biological therapy agents (e.g., therapeutic peptides described in U.S. Pat. No. 6,306,832, WO 2012007137, WO 2005000889, WO 2010096603 etc.). In some embodiments, the at least one additional therapeutic agent is a chemotherapeutic agent.

Specific chemotherapeutic agents include, but are not limited to, cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), cladribine, midostaurin, bevacizumab, oxaliplatin, melphalan, etoposide, mechlorethamine, bleomycin, microtubule poisons, annonaceous acetogenins, chlorambucil, ifosfamide, streptozocin, carmustine, lomustine, busulfan, dacarbazine, temozolomide, altretamine, 6-mercaptopurine (6-MP), cytarabine, floxuridine, fludarabine, hydroxyurea, pemetrexed, epirubicin, idarubicin, SN-38, ARC, NPC, campothecin, 9-nitrocamptothecin, 9-aminocamptothecin, rubifen, gimatecan, diflomotecan, BN80927, DX-8951f, MAG-CPT, amsacnne, etoposide phosphate, teniposide, azacitidine (Vidaza), decitabine, accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, streptozotocin, nimustine, ranimustine, bendamustine, uramustine, estramustine, mannosulfan, camptothecin, exatecan, lurtotecan, lamellarin D9-aminocamptothecin, amsacrine, ellipticines, aurintricarboxylic acid, HU-331, or combinations thereof.

Examples of antimetabolites include 5-fluorouracil (5-FU), 6-mercaptopurine (6-MP), capecitabine, cytarabine, floxuridine, fludarabine, gemcitabine, hydroxyurea, methotrexate, pemetrexed, and mixtures thereof.

Examples of taxanes include accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, and mixtures thereof.

Examples of DNA alkylating agents include cyclophosphamide, chlorambucil, melphalan, bendamustine, uramustine, estramustine, carmustine, lomustine, nimustine, ranimustine, streptozotocin; busulfan, mannosulfan, and mixtures thereof.

Examples of topoisomerase I inhibitors include SN-38, ARC, NPC, camptothecin, topotecan, 9-nitrocamptothecin, exatecan, lurtotecan, lamellarin D9-aminocamptothecin, rubifen, gimatecan, diflomotecan, BN80927, DX-8951f, MAG-CPT, and mixtures thereof. Examples of topoisomerase II inhibitors include amsacrine, etoposide, etoposide phosphate, teniposide, daunorubicin, mitoxantrone, amsacrine, ellipticines, aurintricarboxylic acid, doxorubicin, and HU-331 and combinations thereof.

Examples of immunotherapeutic agents include immune checkpoint inhibitors (e.g., antibodies targeting CTLA-4, PD-1, PD-L1), ipilimumab, 90Y-Clivatuzumab tetraxetan, pembrolizumab, nivolumab, trastuzumab, cixutumumab, ganitumab, demcizumab, cetuximab, nimotuzumab, dalotuzumab, sipuleucel-T, CRS-207, and GVAX.

In any case, the multiple therapeutic agents may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents.

### Kits

The present disclosure also provides kits for selecting a lung cancer patient for treatment with a HER2-targeted therapeutic agent disclosed herein. The kits comprise reagents for detecting HER2 dimerization in a biological sample obtained from the patient. In some embodiments, the reagents for detecting HER2 dimerization include but are not limited to, binding agents (e.g., antibody) specific for HER2, either alone or in combination with binding agents (e.g., antibody) specific for HER3. Additionally or alternatively, in some embodiments, the binding agents are conjugated to one or more detectable labels. In some embodiments, the detectable labels are fluorochromes. In certain embodiments, fluorochromes are selected such so that their excitation/emission spectra facilitate fluorescence resonance energy transfer (FRET) and/or fluorescence lifetime imaging microscopy (FLIM). Optionally, the above described components of the kits of the present technology are packed in suitable containers and labeled for selecting a lung cancer patient for treatment with a HER2-targeted therapeutic agent disclosed herein.

For antibody-based kits, the kit can comprise, e.g., 1) a first antibody, e.g. anti-HER2 antibody; 2) optionally, a second, different antibody (or antibodies), e.g., anti-HER3; 3) optionally, a third antibody, which binds to the first antibody and is conjugated to a first detectable label; and 4) optionally, a fourth antibody, which binds to the second antibody or the second antibodies, and is conjugated to a second detectable label. The kit can further comprise components necessary for detecting the detectable-label, e.g., an enzyme or a substrate.

The kits are useful for selecting a lung cancer patient for treatment with one or more HER2-targeted therapeutic agents disclosed herein based on the detection of HER2 dimerization in a biological sample, e.g., any body fluid including, but not limited to, e.g., serum, plasma, lymph, cystic fluid, urine, stool, cerebrospinal fluid, ascitic fluid or blood and including biopsy samples of body tissue. The biological sample may be Formalin-Fixed Paraffin-Embedded (FFPE) tissue samples, fresh tissue samples or frozen tissue samples. For example, the kit can comprise anti-HER2 antibody, alone or in combination with anti-HER3 antibodies; a means for determining the amount of HER2 dimers in the sample; and a means for comparing the amount of HER2 dimers in the sample with a standard. One or more of the antibodies may be labeled. The kit components, (e.g., reagents) can be packaged in a suitable container. The kit can further comprise instructions for using the kit to select a lung cancer patient based on the detection of HER2 dimerization levels.

The present disclosure also provides kits for the prevention and/or treatment of lung cancer (e.g., HER2 mutant lung cancer), comprising a) reagents for detecting HER2 dimerization in a biological sample; and b) one or more HER2-targeted therapeutic agents disclosed herein.

The above-mentioned components may be stored in unit or multi-dose containers, for example, sealed ampoules, vials, bottles, syringes, and test tubes, as an aqueous, preferably sterile, solution or as a lyophilized, preferably sterile, formulation for reconstitution. The kit may further comprise a second container which holds a diluent suitable for diluting the pharmaceutical composition towards a higher volume. Suitable diluents include, but are not limited to, the pharmaceutically acceptable excipient of the pharmaceutical composition and a saline solution. Furthermore, the kit may comprise instructions for diluting the pharmaceutical composition and/or instructions for administering the pharmaceutical composition, whether diluted or not. The containers may be formed from a variety of materials such as glass or plastic and may have a sterile access port (for example, the container may be an intravenous solution bag or a vial having a stopper which may be pierced by a hypodermic injection needle). The kit may further comprise more containers comprising a pharmaceutically acceptable buffer, such as phosphate-buffered saline, Ringer's solution and dextrose solution. It may further include other materials desirable from a commercial and user standpoint, including other buffers, diluents, filters, needles, syringes, culture medium for one or more of the suitable hosts. The kits may optionally include instructions customarily included in commercial packages of therapeutic or diagnostic products, that contain information about, for example, the indications, usage, dosage, manufacture, administration, contraindications and/or warnings concerning the use of such therapeutic or diagnostic products.

The kit can also comprise, e.g., a buffering agent, a preservative or a stabilizing agent. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present technology may contain a written product on or in the kit container. The written product describes how to use the reagents contained in the kit, e.g., selecting a lung cancer patient for treatment with a HER2-targeted therapeutic agent disclosed herein. In certain embodiments, the use of the reagents can be according to the methods of the present technology.

### EXAMPLES

The present technology is further illustrated by the following Examples, which should not be construed as limiting in any way. The examples herein are provided to illustrate advantages of the present technology and to further assist a person of ordinary skill in the art with preparing or using the compositions and systems of the present technology. The examples should in no way be construed as limiting the scope of the present technology, as defined by the appended claims. The following Examples demonstrate the illustrative methods of for treating lung cancer in a patient harboring a HER2 mutation comprising detecting HER2 dimerization in the patient and administering to the patient an effective amount of ado-trastuzumab emtansine. However, other antibody-drug conjugates comprising an anti-HER2 antibody may be used.

### Example 1: Experimental Materials and Methods

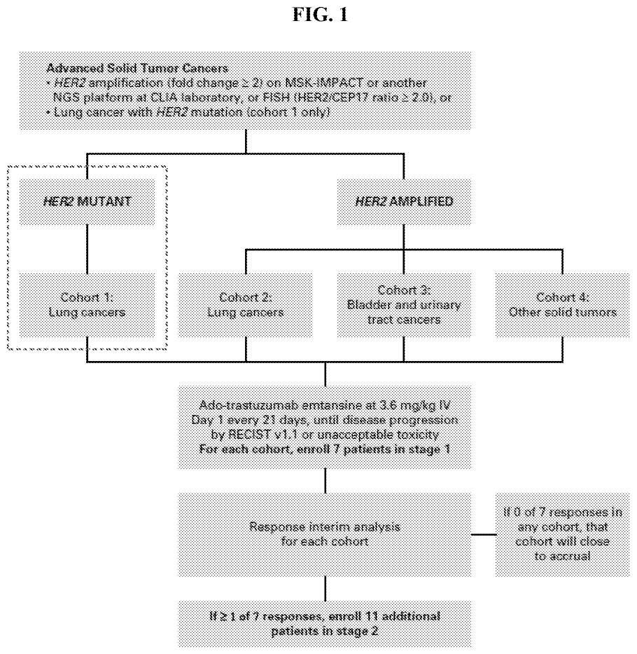

Ado-trastuzumab emtansine (also known as T-DM1) is a HER2-targeted antibody-drug conjugate linking trastuzumab with the anti-microtubule agent emtansine, and is an approved medicine for patients with HER2 amplified or overexpressing metastatic breast cancers. The activity of ado-trastuzumab emtansine was assessed in a cohort of patients with HER2 mutant lung cancers as part of a phase 2 basket trial. Patients with stage 4 or recurrent HER2 mutant lung cancers were enrolled in the cohort, as illustrated in the basket trial scheme in FIG. 1. Other cohorts of this basket trial included HER2 amplified lung, bladder and other solid tumors, as illustrated by FIG. 1. HER2 mutations were identified through next generation sequencing (NGS) including exon 20 insYVMA, insGSP, insTGT, single base pair substitutions L755A, L755S, V777L, V659E, S310F or other likely activating mutations. Other inclusion criteria included age (≥18 years old), Karnofsky performance status (≥70%), measurable disease by Response Evaluation Criteria in Solid Tumors (RECIST) v1.1, and adequate left ventricular, bone marrow, and hepatic function. Patients were eligible regardless of whether they were treatment naïve or had received prior anti-cancer or other HER2-targeted therapy including trastuzumab.

All patients received ado-trastuzumab emtansine (3.6 mg/kg) by intravenous infusion every 21 days until disease progression or unacceptable toxicity was observed. Physical examination and safety assessments were performed every 3 weeks. Tumor assessments using contrast enhanced computed tomography of the chest, abdomen, and pelvis were performed at baseline, week 6, week 12, and every 12 weeks thereafter until disease progression was observed. Brain imaging was not routinely performed unless clinically indicated. Left ventricular ejection fraction measurements by echocardiography or multi-gated acquisition scan were performed at baseline then every 3 months. The primary objective was the determination of overall response rate (ORR; complete plus partial response rate) of ado-trastuzumab emtansine according to RECIST v1.1. Secondary objectives included assessment of progression-free survival (PFS) and toxicity according to the National Cancer Institute Common Terminology Criteria for Adverse Events version 4.1 (NCI CTCAE v4.1). The molecular associations between HER2 mutations, HER2 amplification and HER2 protein expression were examined using different molecular diagnostic assays whenever archival tissue was available. NGS assessed for HER2 mutation and amplification, fluorescence in situ hybridization (FISH) assessed for HER2 amplification, and HER2 protein overexpression was assessed by immunohistochemistry (IHC) and quantitative mass spectrometry. FISH was performed using FDA approved probe sets (PathVysion, Abbott Molecular, Chicago, Ill. and HER2 IQFISH pharmDx, Agilent, Santa Clara, Calif.) and positive HER2 amplification was defined as HER2/CEP17 ratio ≥2.0. HER2 protein by IHC was assessed using the 4B5 Ventana antibody (Ventana Medical Systems, Oro Valley Ariz.). Quantitative HER2 protein by selected reaction monitoring mass spectrometry on fixed-formalin paraffin-embedded tissue was performed using methods previously validated in breast cancers, with ≥740 amol/ug as the cutoff for high HER2 expression (see Nuciforo P., et al., Mol. Oncol., 10:138-147 (2016)).

Statistical Considerations

For each cohort of the basket trial including HER2 mutant lung cancers, a Simon two-stage optimal design was used to determine whether ado-trastuzumab emtansine has sufficient activity to warrant further development in each cohort. Target accrual was a minimum of 7 patients (stage 1) and maximum of 18 patients (stage 1 and 2) in each cohort. The primary endpoint was best confirmed overall response rate (ORR) per RECIST v1.1. The Simon's optimal two-stage design was employed with a multiple testing adjusted type I error rate for each cohort. For a targeted agent where higher response rates were expected, a true ORR of ≤10% was considered unacceptable (null hypothesis), whereas a true ORR of ≥40% merited further study (alternative hypothesis). In the first stage, 7 patients were accrued; if there were no responses observed at interim analysis of the 7 patients in a particular cohort, the cohort was closed. Otherwise, 11 additional patients were accrued for a total of 18 patients. For the overall trial, the null hypothesis would be rejected for each cohort separately if at least 5 responses were observed in each cohort. This design controls type I error rate at 2.7% and generates 89% power for detecting active cohorts. The overall family-wise error rate at the study level was <10%. The exact 95% CI for ORR was calculated using the Clopper-Pearson method. PFS time was estimated by the Kaplan-Meier method. Follow-up time was calculated from the start of treatment to the most recent patient follow-up assessment.

### Example 2: Effects of Ado-Trastuzumab Emtansine Treatment in Patients with HER2 Mutant Lung Adenocarcinomas

Patients

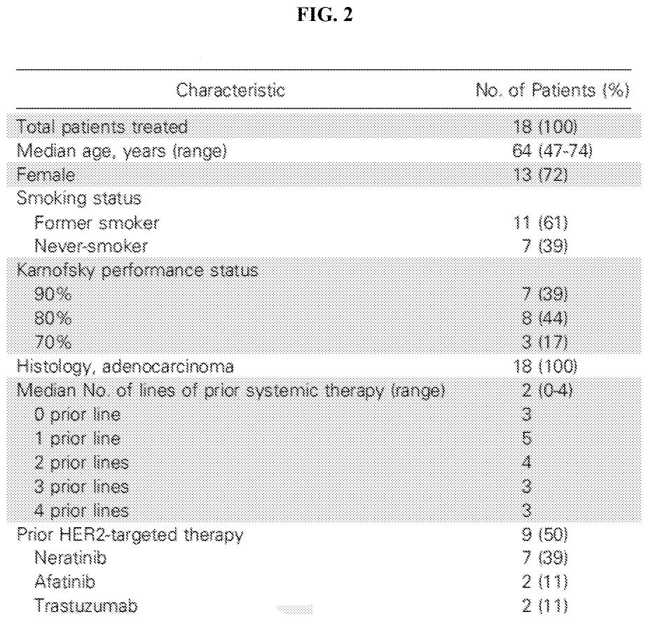

A cohort of 18 patients with metastatic HER2 mutant lung adenocarcinomas was accrued. The median follow-up time was 10 months. Sixteen patients (89%) were identified through MSK-IMPACT (Memorial Sloan Kettering Cancer Center, New York, N.Y.) NGS. Patient characteristics are presented in FIG. 2. The median number of lines of prior systemic therapy was 2 (range, zero to four lines of prior therapy), and 50% of patients had received prior HER2-targeted therapy including neratinib, afatinib and trastuzumab.

Clinical Activity and Safety

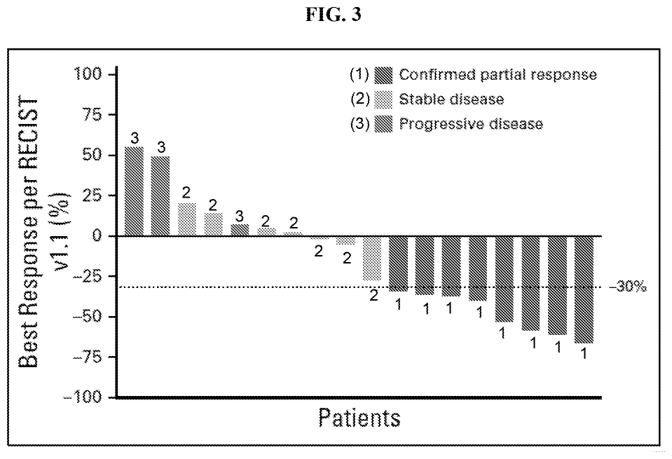

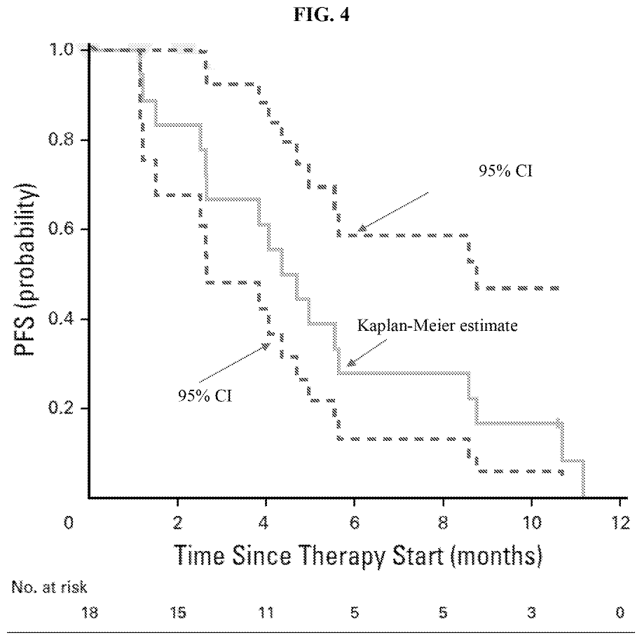

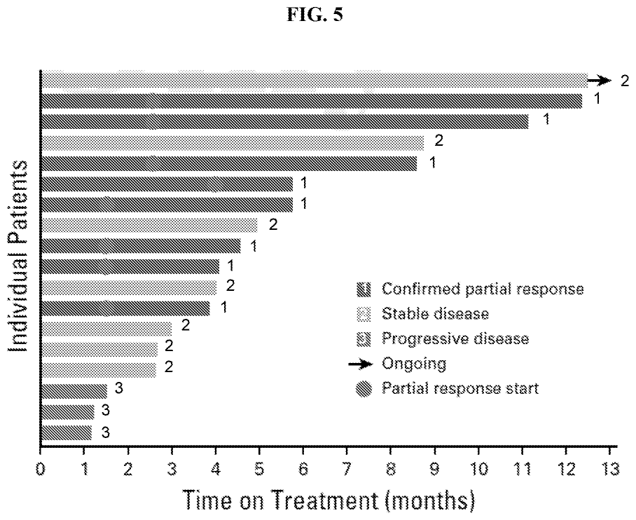

The ORR (all partial and confirmed) was 44% (95% CI, 22-69%) as summarized in FIG. 3, thus rejecting the null hypothesis. Three of 18 (17%) patients had progression of disease as best response. The median PFS for all patients was 5 months (95% CI, 3 to 9 months), and median PFS for the responders was 6 months (95% CI, 4 months to not reached; FIG. 4). The longest PFS observed (11+ months) was in a patient with stable disease as best response with −27% tumor shrinkage (FIG. 5). The median number of cycles of ado-trastuzumab emtansine administered was 6 (range, 2 to 19 cycles). The median duration of response was 4 months (range, 2 to 9 months). The median time to response from start of treatment was 2 months (range, 1 to 4 months). Of the 8 patients with partial responses, 2 were previously untreated, 6 were pretreated with 2 to 4 prior lines of systemic therapy, including 4 patients who received prior HER2-targeted therapy with neratinib and trastuzumab. One patient had previously responded to neratinib plus temsirolimus, but did not respond to trastuzumab plus gemcitabine just before study entry. Three other patients had stable disease on prior neratinib, one of them immediately before study entry. Of the 15 patients who were pretreated with prior systemic therapy, 6 (40%) had responded to ado-trastuzumab emtansine. Only 2 patients had active untreated brain metastases at enrollment, but both patients had progression of disease systemically and in the central nervous system at first response assessment. Seven patients received prior anti-programmed cell death 1 (anti-PD1) immune checkpoint inhibitors, and none responded.

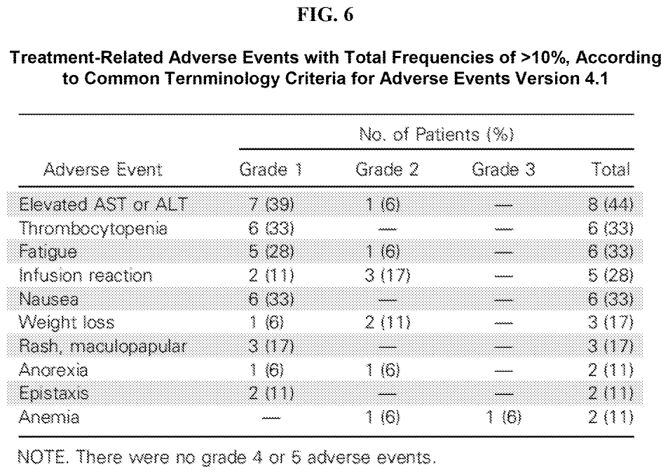

Treatment-related adverse events are summarized in FIG. 6. Adverse events were mainly grade 1 or 2 events, including infusion reactions, thrombocytopenia, and elevations of hepatic transaminases. Infusion reactions characterized by mild rigors, chills, pruritis, and wheezing during treatment occurred in 5 of 18 (28%) patients. These reactions were resolved by slowing the infusion of ado-trastuzumab emtansine and administering antihistamines and did not preclude retreatment. There were no deaths or grade 4 toxicities on study. There were no dose reductions or discontinuations as a result of treatment-related adverse effects.

Biomarker Analyses

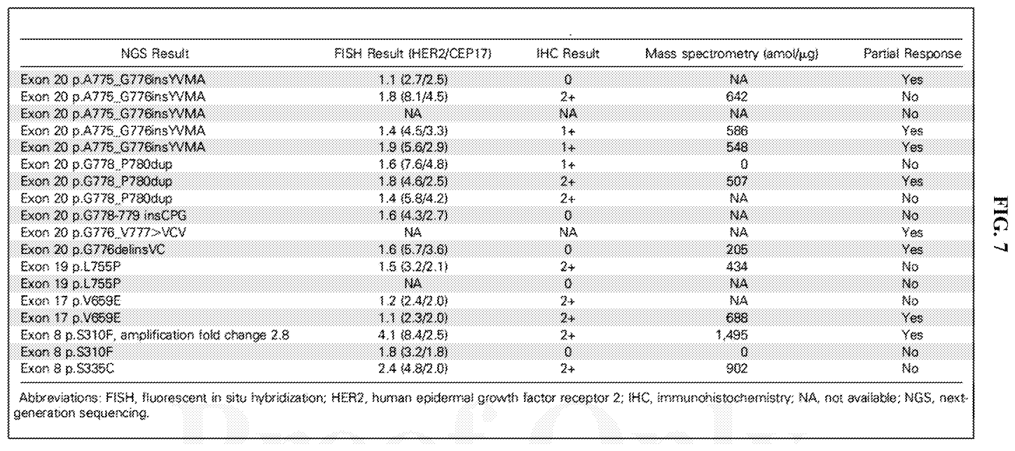

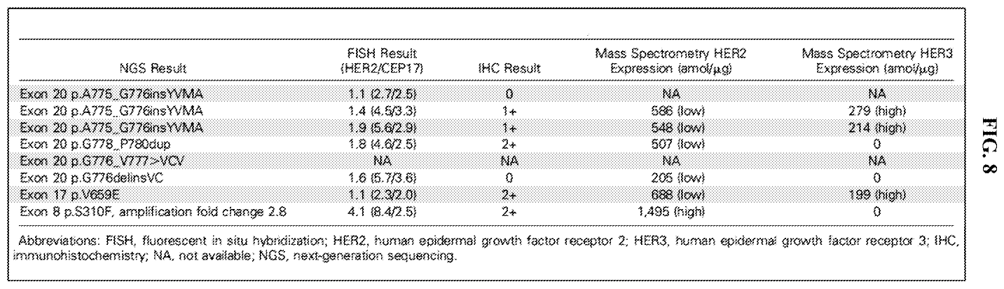

All 18 patients had HER2 activating mutations identified by NGS screening of lung cancer tissue specimens obtained from the patients. HER2 FISH was performed on archival specimens from 15 patients, and HER2 protein by IHC from 16 patients. The results are summarized in FIG. 7. Of note, responders were seen across various HER2 mutation subtypes, including exon 20 insertions, transmembrane and extracellular domain point mutations. Concurrent HER2 amplification was observed in 2 of 18 (11%) patients, both with extracellular domain mutations S310F and S335C, and achieved partial response and stable disease respectively. HER2 IHC ranged from 0 to 2+ among patients both with and without a partial response. There was no association between IHC results and response to ado-trastuzumab emtansine. Quantitative mass spectrometry was performed on 11 patients. For the 9 HER2 mutant lung cancers without amplification, HER2 protein levels were low or non-detectable. The 2 cases with concurrent HER2 amplification showed high HER2 protein levels. Of the patients with partial responses, 5 of 6 tested by mass spectrometry showed low levels of HER2 protein, and the one responder with high HER2 protein had concurrent HER2 gene amplification. Three of 6 patients with partial responses showed increased HER3 expression (FIG. 8).

Ado-trastuzumab emtansine was well tolerated in patients with a comparable side effect profile to that seen in patients with HER2 amplified breast cancers with the exception of a higher than expected rate of grade 1 or 2 infusion reactions compared to the experience in breast cancers as noted in the FDA label (28% vs 1%). In all cases infusion reactions were mild, never required drug discontinuation and were managed by slowing infusions and administering antihistamines. Subsequent events were prevented by the administration of prophylactic antihistamines and acetaminophen.

Treatment with ado-trastuzumab emtansine produced a 44% confirmed partial response rate and a median PFS of 5 months in a largely heavily pre-treated population of patients with advanced HER2 mutant lung cancers, thus achieving the primary endpoint of the study. An additional 39% of patients achieved stable disease, including durable disease control up to 11+ months. These findings are remarkable especially in view of the negative results observed in past clinical trials that targeted HER2 in lung cancers.

These results are also counterintuitive to the conventional theories pertaining to HER2 targeted therapy. Specifically, the initial development of HER2-targeted therapy in lung cancers focused on protein expression by IHC, which was driven in part by observations in breast cancers that trastuzumab binding requires HER2 protein overexpression in order to elicit antitumor activity through inhibition of ligand independent HER2 signaling, receptor internalization, and antibody dependent cell mediated cytotoxicity (Slamon D J et al., N Engl J Med 344:783-92, 2001; Hudis C A, N Engl J Med 357:39-51, 2007). However, HER2 IHC 3+ or HER2 amplification by FISH are much rarer in lung tumors than in breast cancers (2% vs 20%). Further, clinical trials testing the activity of trastuzumab in lung cancers were conducted in tumors with lower levels of HER2 IHC positivity and/or not driven by HER2 signaling. The results of six phase 2 trials of trastuzumab in HER2 IHC positive lung cancers were uniformly negative, and more recent studies of ado-trastuzumab emtansine have again confirmed that HER2 IHC is not the ideal biomarker in lung cancers.

As described above, out of 18 patients with ERBB2-mutant lung cancers, 8 experienced confirmed radiologic partial response to ado-trastuzumab emtansine therapy and, in several additional patients, the disease was controlled over time. Some of these patients were treated for more than 6 months, indicating a relatively prolonged response to ado-trastuzumab emtansine (TDM1). Responders were seen across mutation subtypes. Moreover, of the responding patients, only one had a tumor with concomitant ERBB2 amplification/HER2 overexpression.

Biomarker analysis found concurrent HER2 amplification in 11% of HER2 mutants. Such a small overlap demonstrates that HER2 mutation and amplification are largely separate therapeutic targets. As demonstrated herein, the low or undetectable levels of HER2 protein expression (confirmed by mass spectrometry) among HER2 mutants and the observed responsiveness to ado-trastuzumab emtansine support an alternative mechanism for binding and internalization of the antibody drug conjugate that is independent of HER2 protein overexpression. Without wishing to be bound by theory, it is believed that HER2 activating mutations in lung cancers may exhibit increased dimer formation (e.g., HER2-HER2 homodimers or HER2 heterodimers), which may consequently increase preferential binding and internalization of ado-trastuzumab emtansine (e.g., via increased phosphorylation and receptor ubiquination) to produce antitumor response. For instance, in some embodiments, ado-trastuzumab emtansine bound to mutant HER2 (e.g., V659E and S310F) may be internalized into the cell at a higher rate compared to the wild-type receptor, regardless of the quantity of HER2 protein.

These results demonstrate that the methods of the present technology are useful for treating lung cancer in a subject in need thereof.

### Example 3: Quantitative HER2-HER3 Heterodimerization by Fluorescence Lifetime Imaging Microscopy

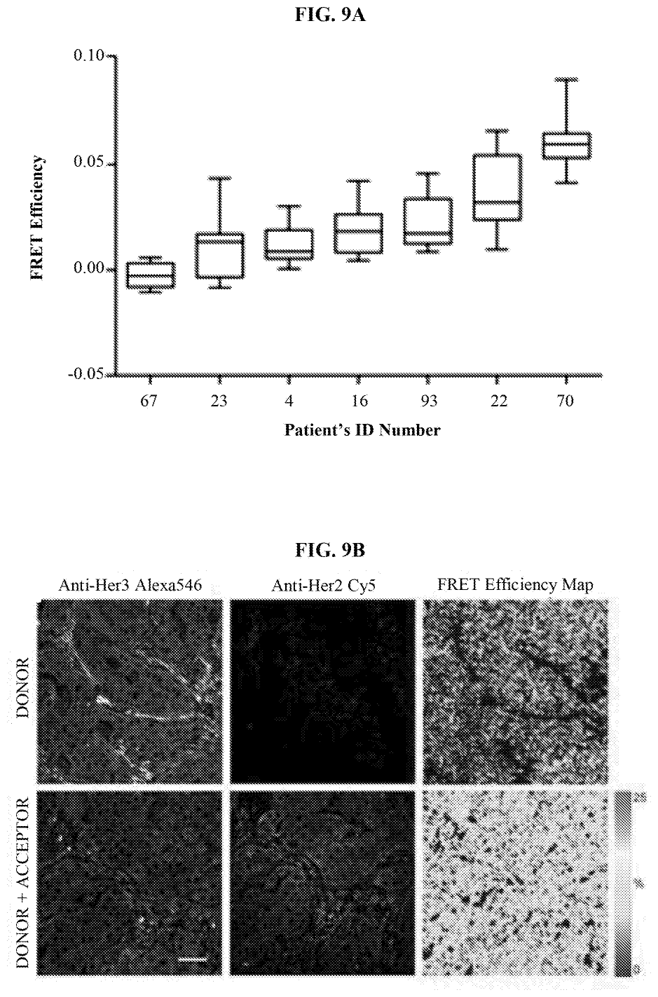

Clinical trial samples were tested for quantitative HER2-HER3 heterodimerization by fluorescence lifetime imaging microscopy-Förster resonance energy transfer (FLIM-FRET) assay. Tissue samples from lung cancer patients were stained with either anti-HER3 Alexa546 antibody (donor), or with the combination of anti-HER3 Alexa546 with anti-HER2 Cy5 antibodies (donor⁺ acceptor). FRET efficiency was calculated according to equation: FRET efficiency=1−tDA/tD, where tDA is the lifetime of the donor in the presence of the acceptor and to is the lifetime of the donor alone. Reduction in the donor lifetime in the presence of the acceptor indicates an interaction between HER2 and HER3 proteins. As shown in FIG. 9A, HER2-HER3 heterodimer formation was evident in several patients. As shown in FIG. 9B, representative FRET images of tissue samples from patient ID number 70 stained with anti-HER3 Alexa546 (donor) and with the combination of anti-HER3 Alexa546 with anti-HER2 Cy5 antibodies (donor⁺ acceptor) demonstrated that the FRET efficiency map showed yellow-red color in donor⁺ acceptor images and blue-green color in donor only images, indicating an interaction between HER2 and HER3 proteins.

In patients with HER2-mutant lung cancers, a positive FLIM-FRET efficiency was found for HER2-HER3 heterodimer formation in three patients tested and one of them had a partial response to ado-trastuzumab emtansine (T-DM1). The heterodimer formation has been shown to be affected by the symmetrical heterodimer interface mutations (Claus et al., eLife 7:e32271 (2018)). In patients with HER2-amplified salivary gland cancers, FLIM-FRET tested positive in 3/3, and all 3 patients responded to T-DM1.

Accordingly, elevated HER2 dimerization levels in lung cancer patients can be used to predict responsiveness to HER2-targeted therapeutic agents, e.g., anti-HER2 antibody drug conjugates. These results demonstrate that the methods of the present technology are useful for treating lung cancer in a subject in need thereof.

### Example 4: Elevated HER2 Dimerization in Lung Cancer Cells Augments Response to Antibody-Drug Conjugate Treatment

Human epidermal growth factor receptor 2 (HER2, ERBB2) mutations occur in a small proportion of solid tumors, from 2% in lung and colon cancer to 3-4% in breast cancer and 8-10% in bladder cancer. This Example demonstrates that responsiveness of lung cancer patients to ado-trastuzumab emtansine therapy can be attributed at least in part to HER2 dimerization. Without wishing to be bound by theory, it is believed that activating mutations of HER2 result in enhanced receptor dimerization and increased baseline phosphorylation, internalization and receptor turnover. The increase in phosphorylation and HER2 internalization is expected to enhance antibody-drug conjugates (ADCs) entrance into the cells, thereby leading to the release of the chemotherapy payload.

The activity of two ADCs (DS-8201 and TDM1) will be tested in HER2-mutant isogenic models. Cell lines expressing several HER2 mutations (HER2 S310F mutation, the 772-775 insertion and the 778-780 insertion, among others) will be established using CRISPR Cas 9 techniques. The endogenous levels of HER2 expressed by these cell lines is expected to be similar to the levels of the receptor expressed by non-amplified/non-overexpressing tumor cells. At least one of the models will be established from a non-tumorigenic cell line, in order to evaluate the transforming activities of these mutations. Such non-tumorigenic cell lines will permit the discrimination of the “clean” contribution of the ERBB2 mutations in increasing ADCs internalization and activity.

In addition to these isogenic models, patient-derived xenografts established from ERBB2-mutant bladder, breast, colon and lung cancers will be employed. These unique in vivo models will be used to test the activity of ADCs. The activity of ADCs in combination with the irreversible HER2 inhibitor neratinib (which enhances HER2 internalization and degradation) will be tested across all the preclinical models described herein. Other models of ERBB2-mutant solid tumors may also be used. HER2 receptor dimerization will be measured by fluorescence resonance energy transfer (FRET; Sekar et al., J Cell Biol. 160(5): 629-633, 2003) in patient samples from HER2 mutant and HER2 amplified tumors.

It is anticipated that HER2 mutant and HER2 amplified cells that respond to ADCs will have higher dimerization rate compared to wildtype HER2 tumors or HER2 mutant/amplified that do not respond to ADCs.

These results demonstrate that the methods of the present technology are useful for treating lung cancer in a subject in need thereof.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A method for selecting lung cancer patients for treatment with a HER2-targeted therapeutic agent comprising:
(a) detecting levels of HER2 dimerization in biological samples obtained from lung cancer patients;
(b) identifying lung cancer patients that exhibit HER2 dimerization levels that are elevated compared to that observed in a healthy control subject or a predetermined threshold; and
(c) administering a HER2-targeted therapeutic agent to the lung cancer patients of step (b), optionally wherein the lung cancer patients are human.

2. The method of claim 1, wherein the lung cancer patients harbor a HER2 mutation selected from the group consisting of exon 20 insYVMA, exon 20 insGSP, exon 20 insTGT, exon 20 insCPG, exon 20 G778_P780dup, exon 20 G776_V777>VCV, exon 20 G776delinsVC, L755A, L755S, L755P, V659E, S310F, and V777L.

3. The method of claim 1, wherein the lung cancer is lung adenocarcinoma, squamous cell lung cancer, large cell lung cancer, or small cell lung cancer (SCLC).

4. (canceled)

5. The method of claim 1, wherein HER2 dimerization comprises HER-HER2 homodimerization and/or HER2-HER3 heterodimerization.

6. The method of claim 1, wherein HER2 dimerization levels are detected via fluorescence resonance energy transfer (FRET), fluorescence lifetime imaging microscopy-fluorescence resonance energy transfer (FLIM-FRET), Western blotting, size exclusion chromatography, analytical ultracentrifugation, scattering techniques, NMR spectroscopy, isothermal titration calorimetry, fluorescence anisotropy, mass spectrometry, fluorescence correlation spectroscopy (FCS), fluorescence recovery after photobleaching, (FRAP), or proximity imaging (PRIM).

7. The method of claim 1, wherein the biological samples are fresh tissue samples, frozen tissue samples, or fixed-formalin paraffin-embedded tissue samples.

8. The method of claim 1, wherein the HER2-targeted therapeutic agent comprises a HER2 antibody-drug conjugate, optionally wherein the HER2 antibody-drug conjugate comprises trastuzumab, pertuzumab, margetuximab, or ertumaxomab.

9. (canceled)

10. The method of claim 8, wherein the HER2 antibody-drug conjugate comprises an anthracycline, a microtubule inhibitor, a mitosis inhibitor, a topoisomerase inhibitor, a DNA damaging agent, a histone deacetylase inhibitor, a kinase inhibitor, a nucleotide analog, an amino acid analog, a vitamin analog, an anti-metabolite, emtansine, deruxtecan, lapatinib, poziotinib, neratinib, or afatinib.

11. (canceled)

12. The method of claim 8, wherein the HER2 antibody-drug conjugate is selected from the group consisting of ado-trastuzumab emtansine (T-DM1), A166, ALT-P7, ARX788, DHES0815A, trastuzumab deruxtecan (DS-8201), DS-8201a, RC48, SYD985, MEDI4276 and XMT-1522.

13. The method of claim 1, wherein the lung cancer patients exhibit HER2 and/or HER3 expression levels that are elevated relative to that observed in a healthy control subject or a predetermined threshold.

14. The method of claim 1, wherein the lung cancer patients exhibit HER2 and/or HER3 expression levels that are comparable to that observed in a healthy control subject or a predetermined threshold.

15. The method of claim 13, wherein the HER2 and/or HER3 expression levels are measured using one or more of mass spectrometry, immunohistochemistry (IHC) or fluorescence in situ hybridization (FISH).

16. A method for treating lung cancer in a patient in need thereof comprising administering to the patient an effective amount of a HER2-targeted therapeutic agent, wherein the patient exhibits HER2 dimerization levels that are elevated compared to that observed in a healthy control subject or a predetermined threshold, optionally wherein HER2 dimerization comprises HER-HER2 homodimerization and/or HER2-HER3 heterodimerization.

17. The method of claim 16, wherein the patient harbors a HER2 mutation selected from the group consisting of exon 20 insYVMA, exon 20 insGSP, exon 20 insTGT, exon 20 insCPG, exon 20 G778_P780dup, exon 20 G776_V777>VCV, exon 20 G776delinsVC, L755A, L755S, L755P, V659E, S310F, and V777L.

18. The method of claim 16, wherein the lung cancer is lung adenocarcinoma, squamous cell lung cancer, large cell lung cancer, or small cell lung cancer (SCLC).

19. (canceled)

20. The method of claim 16, wherein the HER2-targeted therapeutic agent comprises a HER2 antibody-drug conjugate, optionally wherein the HER2 antibody-drug conjugate comprises trastuzumab, pertuzumab, margetuximab, or ertumaxomab.

21. (canceled)

22. The method of claim 20, wherein the HER2 antibody-drug conjugate comprises an anthracycline, a microtubule inhibitor, a mitosis inhibitor, a topoisomerase inhibitor, a DNA damaging agent, a histone deacetylase inhibitor, a kinase inhibitor, a nucleotide analog, an amino acid analog, a vitamin analog, an anti-metabolite emtansine, deruxtecan, lapatinib, poziotinib, neratinib, or afatinib.

23. (canceled)

24. The method of claim 20, wherein the HER2 antibody-drug conjugate is selected from the group consisting of ado-trastuzumab emtansine (T-DM1), A166, ALT-P7, ARX788, DHES0815A, trastuzumab deruxtecan (DS-8201), DS-8201a, RC48, SYD985, MEDI4276 and XMT-1522.

25. The method of claim 20, wherein the patient exhibits HER2 and/or HER3 expression levels that are elevated relative to that observed in a healthy control subject or a predetermined threshold.

26. The method of claim 20, wherein the patient exhibits HER2 and/or HER3 expression levels that are comparable to that observed in a healthy control subject or a predetermined threshold.
